(19) United States
(12) Patent Application Publication
(10) Pub. No.: US 2002/0155448 A1
(43) Pub. Date: Oct. 24, 2002

(54) Asymmetrical PCR amplification

(76) Inventors: Qinyin Cai, Pointe-Claire (CA); Peng Ling, Dorval (CA); Jin-Hao Liu, Dorval (CA)

Correspondence Address: Stephen A. Bent, FOLEY & LARDNER, Washington Harbour, 3000 K Street, N.W., Suite 500, Washington, DC, 20007-5109, US

(21) Appl. No.: 09/825,873

(22) Filed: Apr. 5, 2001

(30) Foreign Application Priority Data
Jan. 26, 2001 (CA) 2,332,610

(51) Int. Cl. C12Q 1/68; C12P 19/34

(52) U.S. Cl. 435/6; 435/91.2

(57) ABSTRACT

A DNA amplification approach, enabling the amplification of DNA targets with unknown sequences, involves successive PCR steps. In a first reaction, linear amplification of the target nucleic acid is achieved using one or more specially designed primers. In the second reaction, additional primers are added and exponential amplification of a double stranded DNA is achieved. Combined with conventional DNA sequencing procedures, this approach can be used for the direct sequencing of genomic DNA, avoiding the need to sub-clone large fragments. With the inventive method, moreover, sequencing information can be gathered in a much less costly and labor-intensive manner than was possible previously.

### FIELD OF THE INVENTION

The present invention relates to a novel process for the amplification of DNA based on Polymerase Chain Reaction (PCR) DNA amplification. More specifically, the present invention is concerned with Asymmetrical PCR Amplification (APA), a process comprising the following steps: (1) the accumulation of single-stranded DNA using a set of specially-designed primers and (2) the amplification of double-stranded DNA. APA may be used in a variety of molecular biological and biotechnological applications, including genomic sequencing projects.

### BACKGROUND OF THE INVENTION

The Polymerase Chain Reaction (PCR), developed in the mid-1980s (Saiki et al. 1985) and disclosed in U.S. Pat. No. 4,683,202, No. 4,800,159, and No. 4,965,188, is a powerful tool for the in vitro amplification of nucleic acid sequences. PCR technology has been widely used in molecular biology for DNA cloning, genomic typing and sequencing. It also has been applied to medical and pharmaceutical research, clinical diagnosis and human DNA finger printing.

The PCR is an in vitro cyclical process involving the enzymatic synthesis of double stranded DNA. In this process, a thermostable DNA polymerase is involved and plays a key role in the amplification of DNA. In addition, a pair of oligonucleotides termed “priners” and four deoxyribonucleotides (dATP, dCTP, dTTP and dGTP) as well as buffer components are involved in the reaction. Both primers are significantly complementary to the template DNA sequences that flank the target DNA region to be amplified.

In each cycle of the PCR, following the denaturing of the double-stranded DNA, the primers anneal to a single strand DNA template at a specified temperature level and initiate the extension, via DNA polymerase, of a new strand, which is complementary to the template strand. After a number of reaction cycles, DNA fragments with the termini defined by the 5′ ends of the primers accumulate exponentially.

A variety of PCR permutations have been developed, since its conception. A common limitation among these variations, however, is the necessity to know the DNA sequences flanking the region of interest. Thus, DNA amplification has been limited to templates having known sequences.

Recently, a method of amplifying a target DNA having an unknown sequence was developed by Chenchik et al (U.S. Pat. Nos. 5,759,822 and 5,565,340). This technology requires several DNA templates to be processed by restriction endonuclease digestion, purified and ligated to an adaptor, prior to being used in the PCR reaction. Thus, this process is both complicated and time-consuming.

Genomic sequencing is the key part of genome projects. It is also the most costly. The Human Genome Project, for example, has spent billions of dollars on sequencing. One reason why it is so costly is that there is no reliable technique which can be used for directly sequencing large genomic DNA inserts. Large genomic DNA clones have to be sub-cloned using a “shotgun” cloning procedure before being sequenced. In shotgun cloning, the large genomic DNA clones are sheared into smaller pieces, typically about 1 kilobase, and sub-cloned into a plasmid or phagemid vector. Subsequently, the colonies are randomly selected, and their cloned DNAs sequenced. Finally, all the sequencing data from the individual clones are compiled using computer programs. Normally, about four to six copies of genome have to be sequenced in order to fill in the gaps of a complete genome assembly. Thus, obtaining sequence information from genomic clones is a laborious and expensive process.

### SUMMARY OF THE INVENTION

The present invention addresses a need for a DNA amplification process that is fast, efficient, accurate and inexpensive, and that accommodates small quantities of DNA.

To these ends, there is provided, in accordance with one aspect of the present invention, a method for amplifying a target nucleic acid in a reaction mixture that also contains at least one Degenerate Random Tagging primer, comprising: (A) performing an asymmetrical PCR, at a low annealing temperature, with the target nucleic acid as a template and the Degenerate Random Tagging primer as an amplification primer; and then (B) adding a specific primer and a tagging primer to the reaction mixture and performing a further PCR at a high annealing temperature. In one embodiment, the asymmetrical PCR comprises approximately 10-20 cycles of amplification. In another, the further PCR comprises approximately 30-50 cycles of amplification. In a preferred embodiment, a temperature of approximately 35° C. to 40° C. is used for a low annealing temperature, with 37° C. being most preferred. Temperatures greater than 50° C. are typically used for a high annealing temperature, and temperatures greater than 55° C. are preferred, while 56° C. is most preferred.

In one embodiment, A DRT primer comprises at least two functionally distinct regions, including, from 5′ to 3′, (1) a tagging primer binding portion and (2) a primary binding portion. In a preferred embodiment, a DRT primer further comprises a bi-nucleotide degenerate sequence portion, located between the tagging primer binding portion and the primary binding portion of the bi-nucleotide degenerate sequence portion. In still another embodiment, a single DRT primer is used in an APA, while in another, a mixture of DRT primers is used. In addition, one or more families of DRT primers can be used in the inventive method.

In addition, the present invention provides isolated polynucleotides encoding particular DRT primers. Also provided is a kit for amplifying target nucleic acids comprising at least one DRT primer, a tagging primer and reagents to effect amplification of nucleic acids.

The APA process may be used in a variety of molecular biological and biotechnological applications. It may be used, for example, for genomic sequencing projects such as the following:

1) BAC clone end sequencing for contig alignment or BAC clone full length sequencing, without the need for subcloning;

2) 5′ RACE or 3′ RACE to clone full-length sequences of cDNA;

3) upstream extensions to clone genomic sequences; and

4) sequencing-based genomic typing.

Additionally, the APA process may be utilized in such applications as sequencing-based virus diagnosis for HIV and crop hybrid identification.

Advantageously, APA allows sequencing information to be gathered in a much less costly and labor-intensive manner than has heretofore been possible. It also requires significantly less quantities of DNA than conventional methods (e.g., ng amounts as opposed to μg quantities of starting DNA).

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples of infections where it will be obviously useful to those skilled in the prior art.

### BRIEF DESCRIPTION OF THE DRAWINGS

FIG. 1 is a schematic representation of Degenerate Random Tagging (DRT) primers used in the APA process. A. DRT primer comprising a tagging primer binding portion and a primary binding portion. B. DRT primer comprising three portions: P1, the tagging primer binding portion; P2, bi-nucleotide degenerate sequence portion; and P3, the primary binding portion. C. A family of DRT primers comprising a bi-nucleotide degenerate sequence portion. The bi-nucleotide degenerate sequence is represented by NN, wherein each N comprises one of four types of deoxynucleotides: dATP, dCTP, dGTP and dTTP. Each nucleotide position of the primary binding portion is represented by R with the position defined with a subscript digit.

FIG. 2 provides a schematic depiction of the APA process. APA involves two subsequent PCR. The first reaction is an asymmetrical PCR performed with a low annealing temperature and a specially designed amplification primer, designated a Degenerate Random Tagging (DRT) primer. Upon completion of the first reaction, two additional amplification primers, i.e., a specific primer and a tagging primer, are added to the reaction mixture. Then, a second PCR is performed at a high annealing temperature.

FIG. 3 is a schematic representation of a BAC (bacterial artificial chromosome) clone insert and flanking DNA sequence of the pecBAC1 vector.

FIG. 4 shows the effect of template concentration on the yield of APA-PCR products. A. T7-flanked end sequence amplification of clone CO2-I17 with different amounts of DNA template using the Le1 DRT primer. The amount of DNA template varied from 0 ng to 10 ng. Lanes 1-6: APA product amplified, respectively, in 0, 0.5, 1.25, 2.5, 5 and 10 ng templates. Lane 7: 1 kb DNA marker. B. T7-flanked end sequence amplification of clone CE3-A1 using the Le1 DRT primer. Lane 1, 1 kb DNA marker and lanes 24: APA product amplified, respectively, in 1.25, 2.5 and 5 ng templates.

FIG. 5 shows the effect of annealing template during the first PCR round on APA products. A. T7-flanked end sequence amplification of clone CE3-A1 using the Le1 or Le2 DRT primer. Lane 1: 1 kb DNA marker; lanes 24: the APA products derived from the Le1 at 35° C., 37° C., and 39° C.; lanes 5-7: the APA product derived from the Le2 amplification, respectively, at 35° C., 37° C. and 39° C. B. T7-flanked end sequence amplification of clone CO2-G14 using the Le1 DRT primer. Lanes 1-3: the APA product amplified with the Le1 respectively at 35° C., 37° C. and 39° C.; lane 4: 1 kb DNA marker. C. M13R-flanked end sequence amplification of clone CO2-E9 with a Lg1, Lg2 and Lg3 primer mixture. Lane 1: 1 kb DNA marker and lanes 2-4: the APA product derived, respectively, from amplification at 35° C., 37° C. and 39° C.

FIG. 6 shows APA of T7- and M13R-flanked end sequences of CE3-A1 using the Le1, Le2 and Le3 DRT primers. Lane 1: Le1, T7; lane 2: Le1, M13R; lane 3: Le2, T7; lane 4: Le2, M13R; lane 5: Le3, T7; lane 6: Le3, M13R; lane 8: 1 kb DNA marker.

FIG. 7 shows APA products of the T7-flanked end of CO2-G14 using the Le1, Le2 or Le3 DRT primer. Lanes 1-3, Le1, Le2 and Le3, respectively; lane 4, 1 kb DNA marker.

FIG. 8 is a comparison of partial sequencing gel images and chromatographs of APA products amplified from the T7-flanked end of the contaminated BAC clone CO2-H16 using DRT primer Le1 (panel A), Le2 (panel B) and a Le1/Le2 mix (panel C).

### DETAILED DESCRIPTION OF THE EMBODIMENTS

The present invention provides a method of amplifying an uncharacterized, target nucleic acid. The method, termed “Asymmetrical PCR Amplification (APA),” involves two subsequent PCR. The first reaction is an asymmetrical PCR performed with a low annealing temperature and a specially designed amplification primer, designated a Degenerate Random Tagging (DRT) primer. Upon completion of the first reaction, two additional amplification primers, i.e., a specific primer and a tagging primer, are added to the reaction mixture. Then, a second PCR is performed at a high annealing temperature.

Since amplification is achieved without knowledge of the sequence of the target nucleic acid, the inventive methods particularly are useful for generating DNA templates for sequencing reactions from uncharacterized nucleic acids. By not requiring sub-cloning of large DNA fragments prior to pre-sequencing amplifications, the present invention significantly reduces the time and cost associated with sequencing genomic clones.

Definitions

The term “polymerase chain reaction” refers to the process of amplifying nucleic acids in vitro disclosed in U.S. Pat. Nos. 4,683,202, 4,800,159 and 4,965,188 and Saiki et al., Science, 230:1350-1354 (1985). Typically, each cycle of amplification comprises three steps: (a) a denaturing step, (b) an annealing step and (c) an extension step. A typical PCR reaction comprises a nucleic acid template, two amplification primers complementary to the two 3′ borders of the duplex segment to be amplified, four deoxyribonucleotides (dATP, dCTP, dTTP and dGTP) and an appropriate buffer.

The term “asymmetric PCR” or “asymmetrical PCR” refers to a PCR in which a single amplification primer is used. As understood in the art, asymmetrical PCR produces linear amplification, as opposed to the geometric amplification achieved by regular PCR.

The term “amplification primer” refers to an oligonucleotide which hybridizes with a nucleic acid template and provides a free 3′ hydroxyl group which is used by DNA polymerase to initiate extension of DNA fragments complementary to the template.

The term “genomic DNA” refers to chromosomal DNA and can include introns. An intron is an intervening sequence. It is a non-coding sequence of DNA within a gene that is transcribed into hnRNA but is then removed by RNA splicing in the nucleus, leaving a mature mRNA which is then translated in the cytoplasm. The regions at the ends of an intron are self-complementary, allowing a hairpin structure to form naturally in the hnRNA.

The term “vector” refers to a DNA molecule, such as a plasmid, cosmid, phagemid, or bacteriophage or other virally-derived entity, which typically has a capability of replicating in a host cell and which is used to transform cells for gene manipulation. Vectors typically contain one or more restriction endonuclease recognition sites at which foreign DNA sequences may be inserted in a determinable fashion without loss of an essential function of the vector, as well as a marker gene which is suitable for use in the identification and selection of cells transformed with the cloning vector. Appropriate marker genes typically include genes that provide various antibiotic or herbicide resistance. A variety of markers are available to the skilled artisan.

Primers

The present invention uses three types of primers: a Degenerate Random Tagging primer, a specific primer and a tagging primer.

Degenerate Random Tagging Primer

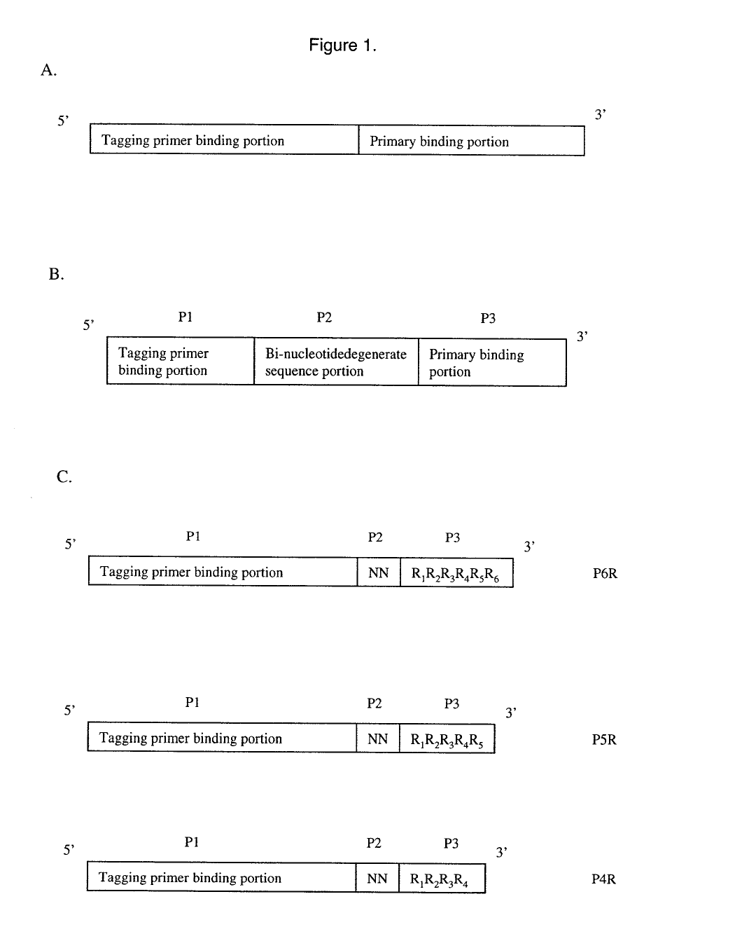

In the asymmetrical step of the amplification, a Degenerate Random Tagging (DRT) primer is utilized. A DRT primer comprises at least two functionally distinct regions portions. From the 5′ to 3′, these include (1) a tagging primer binding portion and (2) a primary binding portion. FIG. 1A provides a schematic representation of a DRT primer. The tagging primer binding portion encodes a position for a later added tagging primer to bind during the second step of DNA amplification. Meanwhile, the primary binding portion comprises an arbitrary sequence of nucleotides ranging in length from approximately four to six nucleotides. The function of the primary binding portion is to bind to any portion of a single-stranded DNA template to which it matches under lower stringency conditions and to initiate extension of DNA fragments complementary to the template.

In a preferred embodiment of the present invention, a DRT primer further comprises a bi-nucleotide degenerate sequence portion, located between the tagging primer binding portion and the primary binding portion. See FIG. 1B. Any one of the four deoxyribonucleotides, i.e., dATP, dCTP, dGTP and dTTP, can occupy either position of the bi-nucleotide degenerate sequence portion. Thus, when a DRT primer comprising a bi-nucleotide degenerate sequence portion is synthesized, sixteen distinct oligonucleotides are produced. Accordingly, the use of a bi-nucleotide degenerate sequence portion expands the number of individual DRT primers utilized in the asymmetric PCR step of the inventive method. Such an expansion increases the probability that at least one of the primers will hybridize to the uncharacterized target nucleic acid within an appropriate distance from one end of the target nucleic acid, e.g., preferably about 1-2 kb.

In another preferred embodiment, a family of DRT primers is used in the asymmetrical PCR. The term “family of DRT primers” refers to two or more DRT primers which differ in the number of nucleotides present in the primary binding portion of the primer. For example, FIG. 1C provides a schematic representation of a family of DRT primers comprising a bi-nucleotide degenerate sequence portion. As illustrated, the family comprises primers P6R, P5R and P4R, whose primary binding portions contain six, five and four nucleotides, respectively. In the figure, the symbol “N” denotes any one of the four deoxyribonucleotides and symbols “R₁-R₆” denote particular nucleotide positions in the primary binding portion designated R₁R₂R₃R₄R₅R₆. Since members possess different nucleotide compositions and melting temperatures, the use of a family of DRT primers expansion further increases the probability that at least one primer will hybridize to the uncharacterized target nucleic acid within an appropriate distance from one end of the target nucleic acid.

In a most preferred embodiment, the asymmetrical PCR utilizes a family of DRT primers comprising a bi-nucleotide degenerate sequence portion. Typically, the primary binding portion of the largest member of such a family contains six nucleotides. The other members of the family are derived by serially deleting the 3′ end of the primer. See FIG. 1C. In another embodiment, two or more families of DRT primers can be used the methods of the present invention.

Specific Primer

The term “specific primer” refers to an oligonucleotide which is designed specifically to hybridize to a particular sequence of a known DNA template. A specific primer is used as an amplification primer in the second PCR of the inventive method. Typically, a specific primer is designed to hybridize to a specific sequence within the vector comprising the uncharacterized target nucleic acid. Such a primer can be designed to bind to the vector on either side of the cloned fragment. Once an end of a target nucleic acid has been elucidated by APA and subsequent sequencing, a specific primer can be designed from the characterized portion of the target nucleic acid and used in subsequent APA.

Tagging Primer

The term “tagging primer” refers to an oligonucleotide which is designed specifically to hybridize to a nucleic acid sequence which is complementary to the tagging primer binding portion of a DRT primer. A tagging primer is used as an amplification primer in the second PCR of the inventive method.

The primers of the present invention can be prepared by direct chemical synthesis using the solid phase phosphoramidite triester method (Beaucage and Caruthers, Tetra. Letts. 22(20):1859-1862 (1981)); an automated synthesizer (VanDevanter et al., Nucleic Acids Res., 12: 6159-6168 (1984)); or the solid support method of U.S. Pat. No. 4,458,066. Chemical synthesis generally produces a single stranded oligonucleotide.

Asymmetrical PCR Amplification

The present application discloses a new process of DNA amplification based on PCR technology. Termed “Asymmetrical PCR Amplification” (APA), this process is suitable for the amplification of selective DNA targets with unknown sequences. Coupled with known DNA sequencing procedures, APA can be used for the direct sequencing of genomic DNA.

The invention provides a method for amplifying a target nucleic acid in a reaction mixture that also contains at least one Degenerate Random Tagging primer, comprising: (A) performing an asymmetrical PCR, at a low annealing temperature, with the target nucleic acid as a template and the Degenerate Random Tagging primer as an amplification primer; and then (B) adding a specific primer and a tagging primer to the reaction mixture and performing a further PCR at a high annealing temperature. Thus, APA comprises two steps: an asymmetrical PCR at low annealing temperature and a second PCR at high annealing temperature.

The first step of APA utilizes an asymmetrical PCR. In this reaction, an uncharacterized, target nucleic acid located within a vector is contacted, under a low annealing temperature, with at least one Degenerate Random Tagging primer. The primer is extended with polymerase to form a double-stranded nucleic acid molecule comprising a first extension product and the target nucleic acid. Next, the first extension products are separated from the target nucleic acids. The contacting, extending and separating steps are repeated approximately 10-20 times, and more preferably 10-14 times.

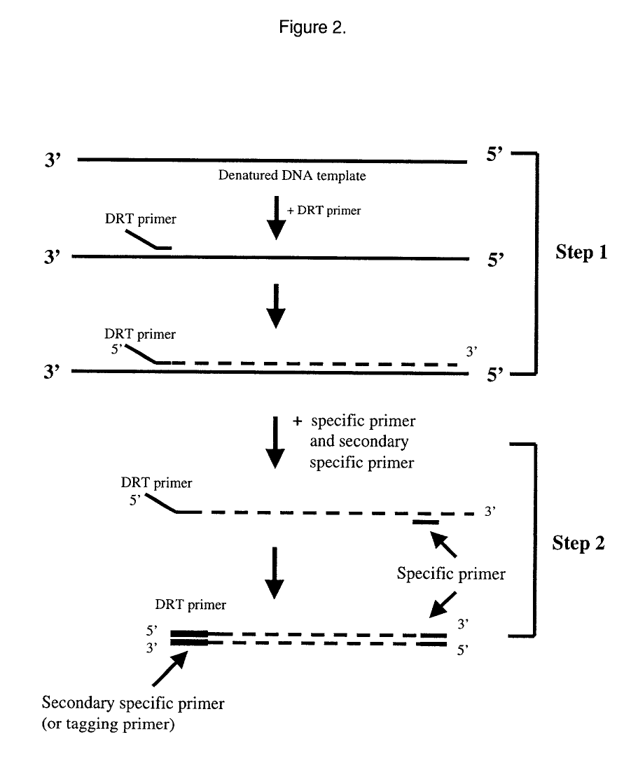

In the second step, a specific primer and a tagging primer are added to the reaction mixture, and a further PCR is performed. In this reaction, the first extension product is contacted, under a high annealing temperature, with the specific primer. The first extension product serves as a template for synthesizing a second extension product. The specific primer then is extended with polymerase to form a double-stranded nucleic acid molecule comprising the first extension product and the second extension product. Afterwards, the first and second extension products are separated. Next, the second extension product is contacted, under a high annealing temperature, with a tagging primer, wherein the second extension product serves as a template for synthesizing a third extension product. The second and third extension products then are separated. The contacting, extending and separating steps are repeated approximately 30-50 times. FIG. 2 provides a schematic representation of APA.

A low annealing temperature is used in the annealing step of the asymmetrical PCR to encourage hybridization between the primary binding portion of a DRT primer and the target nucleic acid. As understood in the art, a low annealing temperature yields a low stringency hybridization and typically refers to temperatures in the range of approximately 30° C. to 45° C. In a preferred embodiment, a temperature of approximately 35° C. to 40° C. is used, and 37° C. is most preferred.

The stringent hybridization conditions resulting from the high annealing temperature utilized in the second PCR ensure that the specific primer and the tagging primer bind specifically to their respective targets and inhibit non-specific amplification. As understood in the art, a high annealing temperature typically is considered to be greater than 50° C. In preferred embodiments, the annealing temperature is greater than 55° C., and 56° C. is most preferred.

The goal of the inventive method is to generate many copies of a double stranded DNA fragment which is bounded on one end by the specific primer and the other end by the tagging primer. As discussed above, a high annealing temperature furthers this goal by reducing non-specific amplification. In addition, formation of “pan” structures by self-end annealing inhibits the undesired amplification of fragments bounded on both ends by a DRT primer. See U.S. Pat Nos. 5,759,822 and 5,565,340.

Uses of APA

APA can be used in a variety of molecular applications For example, the inventive method is useful for 1) BAC clone end sequencing for contig alignment or BAC clone full length sequencing, without the need for subcloning; 2) 5′ RACE or 3′ RACE to clone full-length sequences of cDNA; 3) upstream extensions to clone genomic sequences; and 4) sequencing-based genomic typing. In addition, APA can be utilized in such applications as sequencing-based virus diagnosis of HIV and crop hybrid identification.

The subject invention is particularly useful for genomic bacterial artificial chromosome (BAC) clone end sequencing. The BAC vector has been widely used to construct genomic platforms (BAC genomic libraries). An essential step in BAC genomic library processing is the BAC clone contig alignment. Typically, this is accomplished through the identification of end-sequence overlapping between different clones. Sequence overlapping usually is identified via physical maps based on restriction endonuclease digestion patterns of BAC clone sequences.

Recently, BAC clone end sequencing has been attempted for BAC clone contig alignment. See Venter et al., Nature, 381:364-366 (1996). Although BAC clone end sequencing data is useful for genomic clone contig alignment, two factors have limited the widespread use of direct BAC clone end sequencing. First, a large amount of DNA is needed for each reaction. For example, a 130 kb BAC clone requires 5-10 μg of DNA for each sequencing reaction. Second, the BAC cloning vector is typically a single copy vector, making it hard to extract large amounts of DNA from a BAC clone. The present invention overcomes these limitations. Because of the effectiveness of APA in amplifying the end regions of a BAC clone, the DNA required for sequencing can be generated from less than 20 ng of BAC clone DNA. Accordingly, the inventive method simplifies the process for BAC clone end sequencing and, thereby, reduces the cost associated with processing BAC genomic libraries.

With the present invention, large genomic DNA clones, including BAC clones, can be sequenced directly without sub-cloning. At first, both ends of the genomic DNA insert is amplified via APA using specific primers which hybridize to the vector. Then, the amplified fragments are sequenced using the vector-specific primer. Once the sequences of the amplified products are determined, new specific primers can be designed and used for subsequent amplification and sequencing. Eventually, the entire genomic DNA of a clone can be resolved through primer walking from both ends.

The present invention provides a number of advantages over conventional procedures for primer walking and shotgun clone sequencing. First, the invented process does not require sub-cloning for BAC clone sequencing. Thus, APA avoids the labor and resource costs associated with these procedures. Second, the present invention reduces the required amount of sequencing. With shortgun clone sequencing, many of the sequenced clones contain the same fragment. Thus, the same region of DNA is sequenced multiple times. Since APA-associated sequencing can “walk” along large DNA templates, repetitive sequencing is avoided. Third, APA generates sufficient quantities of DNA for sequencing reactions using a limited amount of BAC clone template DNA (about 1-5 ng). Thus, the present invention minimizes the scale of BAC clone DNA extraction required for sequencing. In contrast, conventional sequencing procedures using BAC clone DNA directly as a template required large quantities of BAC clone DNA, typically 5-10 μg of DNA for each reaction. This requirement made primer walking difficult, if not impossible. Besides solving this problem, APA simplifies the assembly and analysis of sequencing data. Unlike the data from shotgun clone sequencing, sequencing data derived from APA products comes in a known contig order. Thus, sequencing data can be easily assembled and processed.

The present invention also can be used to identify inter-clone contamination in a BAC genomic library. A BAC clone can be tested for contamination by amplifying clone-derived DNA via APA using a family of DRT primers, e.g., Le1, Le2 and Le3. Alternatively, two or more unrelated DRT primers can be used in the amplification reaction. The amplified fragments are sequenced using the vector-specific primer. The sequences then are analyzed for the presence of overlapping bases. Such overlapping is indicative of a mixed sequence and suggests that the sample is contaminated.

To confirm contamination, additional APA reactions using single DRT primers are performed on DNA derived from the clone of interest. A separate APA is performed with each DRT primer utilized in the initial APA. The amplified fragments are sequenced, and the resulting sequences are compared. If sequence analysis reveals two or more sequences, the clone has been contaminated.

The present invention further concerns kits that are specially packaged for BAC clone sequencing, including end sequencing and full range sequencing. The kit may optionally include reagents required for performing APA and the subsequent sequencing reactions of BAC clones, such as Taq DNA polymerase and its cofactors, optimal buffer components, various APA DRT primers, deoxyribonucleotide-5′-triphosphates and deoxy-deoxyribonucleotides-5′-triphosphates. The kit can also provide BAC universal vector specific primers such as T7 and M13-reverse primers. Alternatively, the end-users can utilize their own specific primers depending on the genomic DNA or types of BAC vector. One control BAC clone DNA sample also can be involved in the kit and can be used as a positive control for the APA reaction. The kit can be used with any type of sequencer. A variety of thermostable DNA polymerases from different sources can be used with the present invention. These include Taq DNA polymerase from Thermus aquaticus, Tth DNA polymerase from Thermus thermophilus and pfu DNA polymerase from Pyrococcus furiosus. These enzymes are commercially available. Alternatively, the thermostable DNA polymerases from cloned gene expression can be used with the present invention.

### EXAMPLE 1

### Asymmetrical PCR Amplification of Unknown Sequences in Chicken BAC Clones

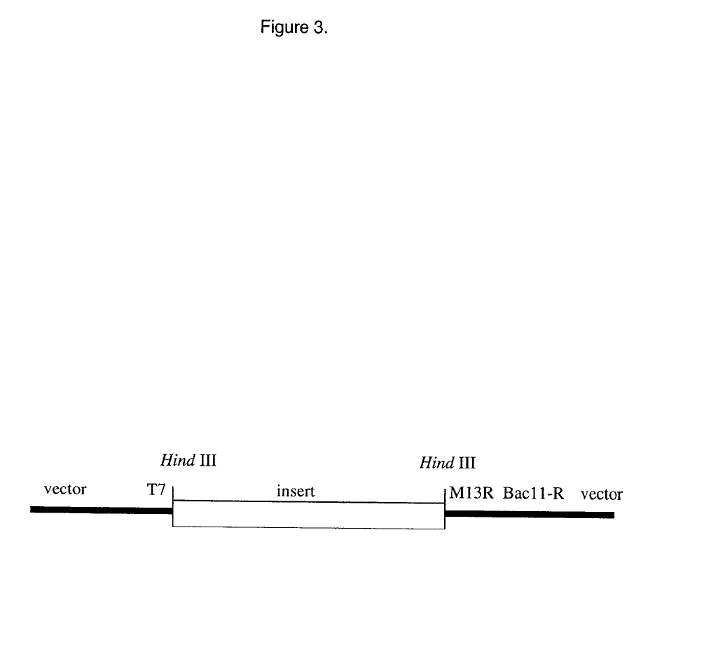

Experiments were carried out to amplify the end sequences of BAC clones using the APA technology disclosed in the current application. BAC clones CE3-A1, CO2-E9, CO2-I17 and CO2-G14 were randomly chosen from a chicken BAC library (Bio S&T Inc.) constructed using plasmid vector pecBAC1. These clones have insert sizes from 80 to 110 kb that were ligated into the Hind III site of the MCS region of pecBAC1. The T7 promoter primer sequence (5′-GTAATACGACTCACTATAGGGC-3′) and the M13 reverse M13R primer sequence (5′-GGATAACAATTTCACACAGG-3′) flanked the inserts (FIG. 3). The T7 primer was used as a specific primer for the amplification of T7-flanked end of inserts using the APA process, while the Bac11-R primer (5′-GAGTTAGCTCACTCATT AGGCAC-3′) next to M13R was used for the amplification of the M 13R-flanked end sequence.

Plasmid DNA was extracted from 5 mls of overnight culture of E. coli DH10B cells harbouring a selected BAC clone. The cell pellet was resuspended in 1 ml of cold GTE buffer (50 mM glucose, 25 mM Tris, pH 8.0 and 10 mM EDTA). The cell suspension was mixed gently with 2 ml of lysis solution (0.2 M NaOH and 1% SDS) and left at room temperature for 5 min. Then, 1.5 ml KacF (3 M K-acetate, 1.8 M formic acid) was added, mixed gently and left on ice for 10 min. After centrifugation at 12,000× g at 4° C. for 10 min, the supernatant was mixed with an equal volume of isopropanol and left at room temperature for 15 min. Plasmid DNA was pelletted by centrifuging as before. The pellet was air dried for 5 min and dissolved in 1 ml TE (50 mM Tris, 10 mM EDTA, pH 8.0). To remove RNA from the sample, 15 μl of 10 mg/ml RNase was added and incubated at 37° C. for 30 min. Then, DNA was extracted with phenol/chloroform and precipitated with ethanol. The concentration of plasmid DNA was determined on a 1% agarose gel stained with ethydium bromide using lambda DNA (Stratagen) as standard.

Table 1 lists the sequences of the two DRT primer families (Le and Lg series) and the tagging primer (EndSeq-T) used. All of the DRT primers comprise a primary binding portion at their 3′ ends (bold letters), a bi-nucleotide degenerate region and a tagging primer binding portion (underlined). Each primer family contains three members. The primary binding portions of Le1 and Lg1 were determined arbitrarily. The other two members in each family resulted from a serial deletion of one deoxyribonuleotide from the 3′ end of Le1 and Lg1, respectively.

In the first step of the APA process, each APA reaction was performed in a total volume of 45 μl reaction mix which contained 60 nmol dNTP, 15 p mol DRT primer, 0.5-10 ng DNA and 4 U Taq DNA polymerase (QIAGEN) in 1× reaction buffer. After 4 min denaturation at 94° C., the reaction was thermocycled for 14 cycles at 94° C. for 45 sec, 37° C. for 50 sec, and 72° C. for 1 min in PTC-200 Thermal Cycler (MJ Research). After the first step of APA, 5 μl of specific primer cocktail containing 15 p mol each of EndSeq-T and T7 or Bac11-R in 1× Taq buffer was added. The PCR in this step was performed as follows: 36 cycles at 94° C., 45 sec, 56° C., 50 sec and 72° C., 1 min, following by 72° C. for 10 min. The PCR product was purified using QIAprep Spin Miniprep Kit (QIAGEN). An aliquot of the samples was loaded onto a 1% agarose gel. All experiments were repeated at least once.

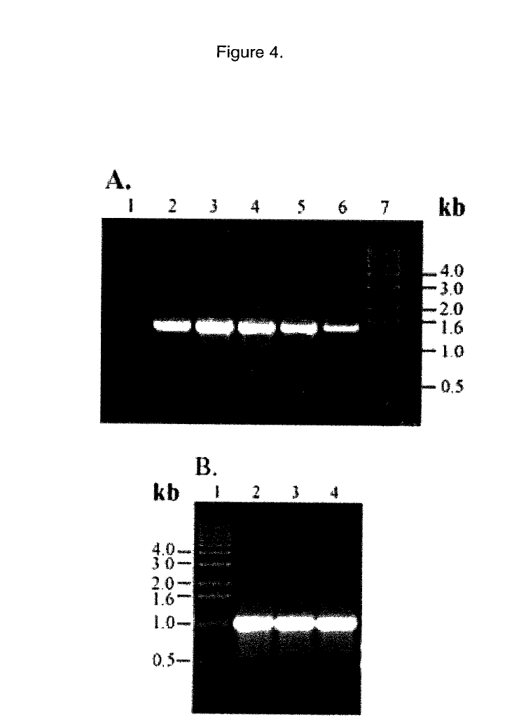

FIG. 4A shows the APA product of the T7 end of clone CO2-I17 using the Le1 as a DRT primer and the T7 primer as a specific primer. A dominant DNA fragment of about 1.5 kb in size was observed for all concentrations of CO2-I17 DNA. The results indicate that the highest amount of product was obtained with 1.25 ng template DNA. For clone CE3-A1, Le1 and the T7 primer amplified a DNA fragment of about 1 kb. No significant difference was observed in yield for template DNA from 1.25 to 5 ng (FIG. 4B).

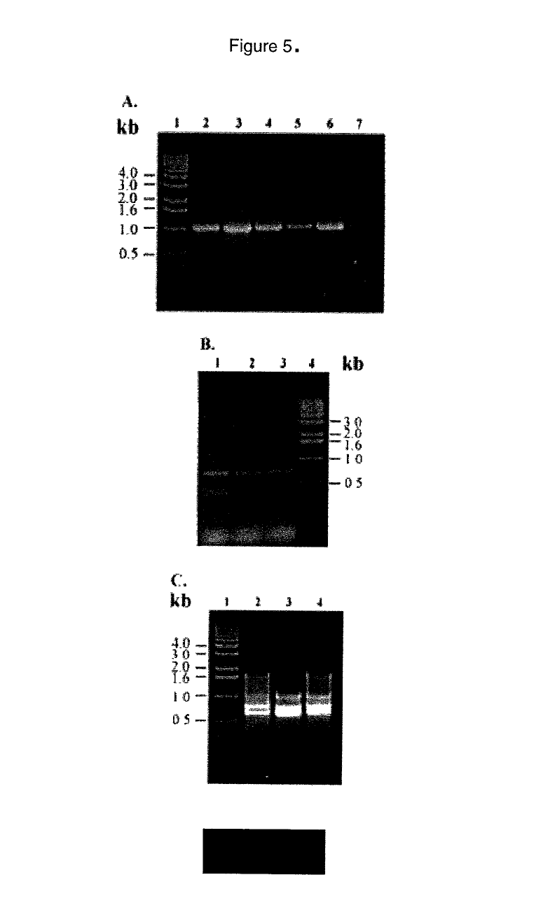

The effect of annealing temperature during the first step APA was studied using clones CE3-A1 T7 end and CO2-G14 T7 end, as well as CO2-E9 M13R end. The T7 end of the inserts in clones CE3-A1 and CO2-G14 were evaluated by using the T7 primer as a specific primer. The M13R end of the insert in clone CO2-E9 was evaluated by using the M13R primer as a specific primer. FIG. 5A demonstrated that 37° C. was the most efficient temperature for DRT primers Le1 and Le2, among the temperatures tested. In the case of Le2, an increase or decrease of 2° C. in annealing temperature not only reduced the yield of products but also reduced the specificity of the APA. Experiments with clones CO2-G14 and CO2-E9 also showed that 37° C. was more specific for T7 end APA than 35° C. and 39° C. (FIGS. 5B and 5C).

### EXAMPLE 2

### Length Effect of Primary Binding Portion of a DRT Primer on APA

Experiments were carried out to examine the effect of the length of the 3′-end primary binding portion of DRT primers on APA and end-sequencing of BAC clones. BAC clones CE3-A1, CO2-E9, CO2-I17 and CO2-G14 were chosen randomly as APA DNA templates from a chicken BAC library (Bio S&T, Inc). These clones comprised inserts of 80, 100, 80, and 110 kb, respectively. Plasmid DNA was extracted using the process described in Example 1. End sequences were amplified by the APA procedures described in Example 1. The amount of template DNA was 2 ng, and the annealing temperature for the first round PCR was 37° C. The T7 primer was used for the APA reaction and for the sequencing of T7-flanked end of clones. To sequence the M13R primer-flanked end of the inserts, the Bac11-R primer was used for the APA reaction and the M13R primer was used for sequencing.

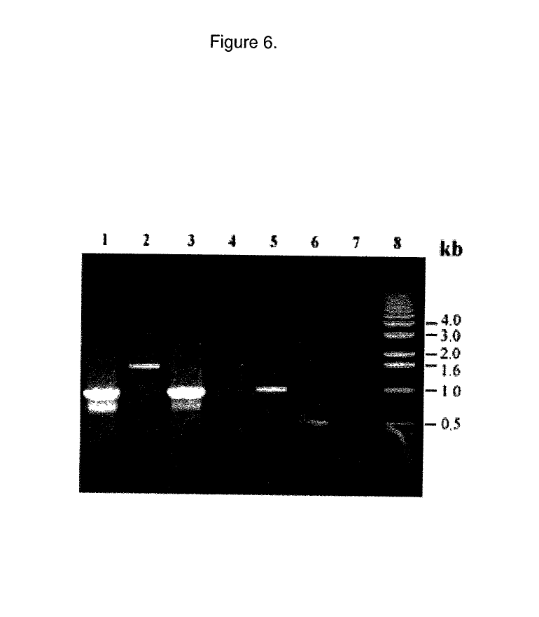

When the DRT primers in the Le family were used in conjunction with the T7 primer to amplify the T7-flanked end of the CE3-A1 insert, a major DNA fragment of about 1 kb in size was obtained for each of the Le1, Le2, and Le3 primers (FIGS. 5 and 6). However, the yield was lower with the Le3 primer. A high sequence resolution was obtained using Le1 and Le2. A similar sequencing result was obtained with Le2. However, the quality of the sequencing resolution with Le3 was relatively poor. Some ambiguity in base calling was found in the sequencing data. This may be due to interference of some shorter fragments amplified by Le3. For example, the Le3 sequence also bound to the position denoted by nucleotide 102, causing noisy signals in this region. A similar result was achieved with Le1 in the amplification and sequencing of the M13R-flanked end sequence. A dominant band of about 1.5 kb in size was shown in the DNA sample amplified with Le1 (FIG. 6). Faint dominant bands along with some shorter bands were shown in the samples amplified with Le2 and Le3, respectively. The sample generated with Le1 resulted in high quality sequencing data, readable up to 1 Kb, while the samples generated by the other two members (Le2 and Le3) showed low sequencing resolution readable to about only 470 bp (data not shown).

Study of other clones has demonstrated that the DRT primer containing 6 random nucleotide sequences resulted in better PCR amplification efficiency and sequencing resolution than the other two members with shorter random sequences. The Le1 primer demonstrated the best performance sequencing resolution, followed by the Le2 primer, with some minor non-specific stops inside the sequence. The Le3 primer showed the poorest sequencing resolution with a strong stop inside the sequence. Accordingly, these results suggest that a DRT primer comprising a six-nucleotide random sequence in its primary binding portion is the ideal candidate for use as a single primer in APA for BAC clone end sequencing.

APA using a combination of DRT primers also was evaluated using the above clones. The following mixtures were evaluated: Le1/2, Le1/2/3, and Le1/Lg1. In most cases, mixed primers Le1/2 and Le1/Lg1 produced results comparable to those achieved by the Le1 primer alone. However, the combination of Le1/2/3 showed poor APA performance. In some cases, the mixed primer Le1/Lg1 successfully amplified sequences that could not be amplified with the Le1 primer or Lg1 primer alone (data not shown). Accordingly, this suggest that the mixed primers of the P6RIP5R or P6R/P6R type (see FIG. 1C) may best ensure the successful amplification of BAC clone end sequences.

### EXAMPLE 3

### Use of APA for BAC Clone End Sequencing

To evaluate the potential of APA for use in BAC clone end sequencing, DNA templates for sequencing reactions were prepared from a population of chicken BAC clones. Chicken BAC clones were randomly selected from a chicken BAC library (Bio S&T Inc.) and amplified using APA. Two DRT primer families were evaluated: Le1/2 and Lg1/2. The individual sequences of each primer in the Le primer series and the Lg primer series are presented in Table 1. The chicken BAC clones used for this test are listed in Table 2. All the DNA samples were amplified by APA using two mixed DRT primers, Le1/2 and Lg1/2, in the manner described in Example 1. The APA amplified product was sequenced using a PCR sequencing kit purchased from Interscience and using the IRD800-labeled T7 and M13R primers. The sequences were resolved in LiCOR 4200L sequencer. All sequencing reactions were repeated at least once. The results are summarised in Table 3. The sequencing data had an accuracy of 99% (based on two repeats). These results demonstrate the effectiveness of APA in amplifying the end sequences of BAC clones.

### EXAMPLE 4

### Identification of BAC Clone Contamination

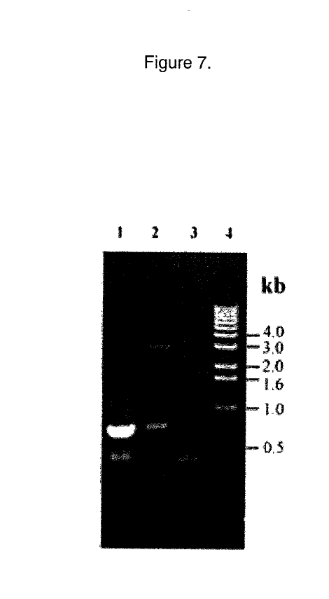

To further determine the effect of the length of the primary binding portion of a DRT primer on APA and end-sequencing, the T7-flanked end of CO2-H16 was amplified by APA using either a Le1, Le2 or Le3 DRT primer. (Sequences provided in Table 1.) A T7 promoter primer was used as the specific primer in the second step of the APA. The reaction conditions described in Example 1 were utilized. The photographed gel in FIG. 7 shows that each DRT primers produced a different number of amplification products. Subsequent sequencing analysis revealed that Le2 and Le3 produced identical amplification products, which differed from those generated by Le1. (Data not shown).

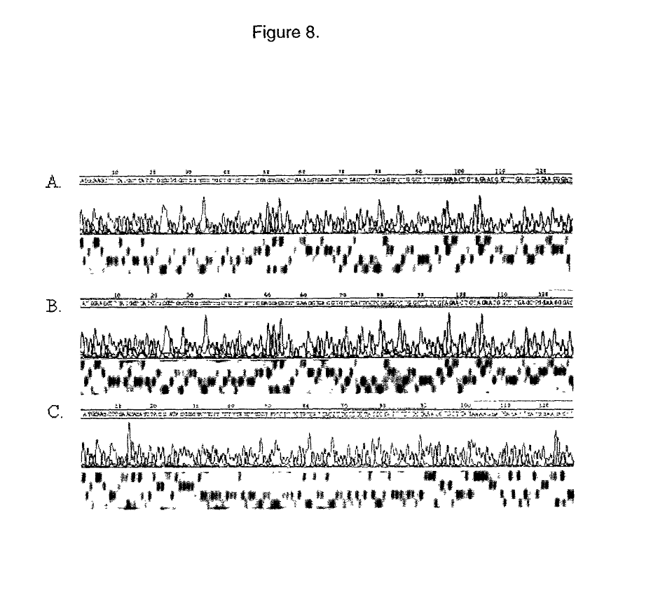

APA also was performed using a mixture of Le1, Le2 and Le3 primers. Sequencing analysis of the resulting amplification demonstrated that the mixture of primers yielded a mixed sequence. Further examination of sequencing gel images derived from APA using both Le1 and Le2 revealed a mixed sequence. The dominant bands of the mixed sequence matched the sequence revealed by Le1, and faint bands corresponding to the Le2 sequence also were observable (FIG. 8). This suggests that the CO2-H16 clone may have been contaminated by one other BAC clone in the library. To further evaluate the possibility of contamination, another DRT primer, Lg1, was used to amplify the same region of CO2-H16 DNA, using the above-referenced reaction conditions. The sequencing data obtained from the Lg1 product was identical to that of the Le2 and Le3, confirming the existence of two different BAC end sequences in the CO2-H16 sample. These results suggest that APA can be used to identify BAC clone contamination.

### EXAMPLE 5

### Use of APA in Primer Walking

To elucidate additional sequence information, beyond that obtained from one round of APA and sequencing, new specific primers were designed for BAC clones Ce3-A1 and CO2-I17. The new specific primers, referred to as walking primers, were designed using the sequences elucidated in previous BAC end sequencing. Primer Ce3-A1T7-2 (5′ CAGGTGTGTGAGTAGAGTTTAG 3′) was selected from nt 750-771 of the sequence of the Ce3-A1 T7-flanked end. Primer Ce3-A1 R2 (5′ GAAAATAGCCAGAGCATCACAGGGC 3′) was selected from nt 700-724 of Ce3-A1 M13R-flanked end. Primer CO2-I17T7-2 (5′ GACGTAACGTGGCAGTGCAA 3′) was designed to match the region of nt 684-703 of CO2-I17 T7-flanked end sequence. APA and sequencing reactions were as described in Examples 1 and 2. IR800-labeled walking primers were used for sequencing.

The use of walking primers in APA, and subsequent sequencing of the amplification products, proved to be an effective means for elucidating additional sequences of uncharacterized cloned inserts. The process, referred to as primer-walking sequencing, was successful for Ce3-A1, with primer Ce3-A1 R2, and CO2-I17, with primer CO2-I17T7-2. Primer walker sequencing using Ce3-A1 R2 showed a 750 nt sequence that extended from nucleotide 747 of the Ce3-A1 M13R-flanked end. Primer walker sequencing using CO2-I17T7-2 revealed a near 800 nt sequence that extended from nucleotide 710 of the CO2-I17 T7-flanked end sequence. Primer walker sequencing using Ce3-A1T7-2, however, was not as successful. The sequencing signal was very weak, and a very strong stop was observed after 300 nt of new sequence. These poor results may be attributable to unusual secondary structure of the DNA template of clone Ce3-A1 in the region near the T7-flanked end sequence.

TABLE 1
Sequences of the DRT primers and the tagging primer
(End Seq-T) used in the current invention

| Primer | Sequence |
|---|---|
| Le1 | 5′-GAGCACCTGTAGACCTCCNNCTACTG-3′ |
| Le2 | 5′-GAGCACCTGTAGACCTCCNNCTACT-3′ |
| Le3 | 5′-GAGCACCTGTAGACCTCCNNCTAC-3′ |
| Lg1 | 5′-GAGCACCTGTAGACCTCCNNCACGCA-3′ |
| Lg2 | 5′-GAGCACCTGTAGACCTCCNNCACGC-3′ |
| Lg3 | 5′-GAGCACCTGTAGACCTCCNNCACG-3′ |
| EndSeq-T(tagging primer) | 5′-GAGCACCTGTAGACCTCC-3′ |

TABLE 2
List of randomly selected chicken BAC clones used for end
sequencing test using APA procedure

| | Estimated | | Estimated |
|---|---|---|---|
| Name of | insert size | Name of | inserted size |
| BAC clone | (kb) | BAC clone | (kb) |
| CE3A1 | 80 | CF1X1 | 65 |
| CE3A2 | 70 | CF1X2 | 72 |
| CE3A4 | 100 | CF1X3 | 110 |
| CE3A5 | 65 | CF1X4 | 65 |
| CE3A6 | 102 | CF1X5 | 130 |
| CE3A8 | 60 | CF1X6 | 50 |
| CE3A15 | 100 | CF1X8 | 70 |
| CE3A16 | 100 | CF1X9 | 50 |
| CE3A17 | 145 | CF1X10 | 60 |
| CE3A18 | 70 | CF1X11 | 130 |
| CE3A19 | 130 | CF1X12 | 100 |
| CE3A21 | 100 | CF1X13 | 57 |
| CE3A22 | 110 | CF1X16 | 85 |
| CO2E9 | 90 | CF1X65 | — |
| CO2F12 | 70 | CF1X67 | 60 |
| CO2G13 | 80 | CF1X71 | 70 |
| CO2G14 | 160 | CF1X73 | 90 |
| CO2H15 | 75 | CF1X74 | 50 |
| CO2I17 | 70 | CF1X75 | 80 |
| CO2J19 | 60 | CF1X76 | 80 |
| CO2J20 | 60 | CF1X77 | 90 |
| CF1G11 | 140 | | |

TABLE 3
The sizes of the BAC clone end sequences resolved through the
currently invented APA technology using two mixed DRT primers:
Le1/2 and Lg1/2

| Name of | | Size of the resolved end sequence | |
|---|---|---|---|
| BAC clone | DRT primer | T7-flanked end (bp) | M13-flanked end (bp) |
| CE3A1 | Le1/2 | 970 | 1100 |
| CE3A2 | Le1/2 | — | 1192 |
| | Lg1/2 | 1253 | 784 |
| CE3A4 | Le1/2 | 626 | 442 |
| | Lg1/2 | 817 | 720 |
| CE3A5 | Le1/2 | 100 | 329 |
| | Lg1/2 | 186 | 1121 |
| CE3A6 | Le1/2 | — | 920 |
| | Lg1/2 | 411 | 954 |
| CE3A8 | Le1/2 | — | 813 |
| | Lg1/2 | 833 | 384 |
| CE3A15 | Le1/2 | 168 | 718 |
| | Lg1/2 | 793 | 726 |
| CE3A16 | Le1/2 | — | 536 |
| | Lg1/2 | 875 | 524 |
| CE3A17 | Lg1/2 | 999 | 852 |
| CE3A18 | Le1/2 | 1122 | 1143 |
| | Lg1/2 | 457 | — |
| CE3A19 | Lg1/2 | 851 | 654 |
| CE3A21 | Le1/2 | — | 1160 |
| | Lg1/2 | — | 454 |
| CE3A22 | Le1/2 | 730 | 1156 |
| CO2E9 | Le1/2 | 1100 | 130 |
| CO2F12 | Le1/2 | 740 | — |
| CO2G13 | Le1/2 | 735 | 545 |
| CO2G14 | Le1/2 | 690 | 240 |
| CO2H15 | Le1/2 | 972 | 1000 |
| CO2I17 | Le1/2 | 1200 | 850 |
| CO2J19 | Le1/2 | 510 | 750 |
| | Lg1/2 | 490 | 1165 |
| CO2J20 | Lg1/2 | 300 | 800 |
| CF1G11 | Lg1/2 | 300 | — |
| CF1X1 | Lg1/2 | 572 | 606 |
| CF1X2 | Le1/2 | 735 | 150 |
| CF1X3 | Le1/2 | 884 | 1307 |
| CF1X4 | Le1/2 | 174 | 494 |
| CF1X5 | Le1/2 | 312 | — |
| CF1X6 | Le1/2 | 706 | — |
| CF1X8 | | 710 | — |
| CF1X9 | Le1/2 | 569 | 914 |
| CF1X10 | Le1/2 | 707 | — |
| CF1X11 | Le1/2 | 682 | — |
| CF1X12 | Le1/2 | 697 | — |
| CF1X13 | Le1/2 | — | 649 |
| CF1X16 | Le1/2 | — | 1054 |
| CF1X65 | Le1/2 | 457 | — |
| CF1X67 | Lg1/2 | 322 | 207 |
| CF1X71 | Lg1/2 | 288 | — |
| CF1X73 | Le1/2 | 388 | — |
| | Lg1/2 | — | 392 |
| CF1X74 | Le1/2 | 350 | — |
| CF1X75 | Lg1/2 | 507 | 166 |
| CF1X76 | Le1/2 | 275 | 324 |
| CF1X77 | Le1/2 | 515 | 104 |

Although the present invention has been described hereinabove by way of preferred embodiments thereof, it can be modified, without departing from the spirit and nature of the subject invention as defined in the appended claims.

What is claimed is:

1. A method for amplifying a target nucleic acid in a reaction mixture that also contains at least one Degenerate Random Tagging primer, comprising:
(A) performing an asymmetrical PCR, at a low annealing temperature, with said target nucleic acid as a template and said Degenerate Random Tagging primer as an amplification primer; and then
(B) adding a specific primer and a tagging primer to said reaction mixture and performing a further PCR at a high annealing temperature.

2. A method according to claim 1, wherein said asymmetrical PCR comprises approximately 10-20 cycles of amplification.

3. A method according to claim 1, wherein said further PCR comprises approximately 30-50 cycles of amplification.

4. A method according to claim 1, wherein said low annealing temperature is approximately 37° C. and said high annealing temperature is approximately 56° C.

5. A method according to any of claims 1 to 4, wherein said DRT primer comprises:
i) A tagging primer binding portion;
ii) A bi-nucleotide degenerate sequence portion; and
iii) A primary binding portion.

6. A method according to any one of claims 1 to 5, wherein said DRT primer is selected from the group consisting of Le1, Le2, Le3, Lg1, Lg2 and Lg3.

7. A method according to any one of claims 1 to 5, wherein a mixture of DRT primers is used.

8. A method according to claim 7, wherein said DRT primers of said mixture are selected from the group consisting of Le1, Le2, Le3, Lg1, Lg2 and Lg3.

9. A method according to any one of claims 1 to 8, wherein said specific primer consists of a nucleotide sequence of 25-30 bases that is complementary to a known sequence of the DNA to be amplified.

10. A Le1 DRT primer having the sequence of Seq. ID No. 1.

11. A Le2 DRT primer having the sequence of Seq. ID No. 2.

12. A Le3 DRT primer having the sequence of Seq. ID No. 3.

13. A Lg1 DRT primer having the sequence of Seq. ID No. 4.

14. A Lg2 DRT primer having the sequence of Seq. ID No. 5.

15. A Lg3 DRT primer having the sequence of Seq. ID No. 6.

16. Use of the process of any one of claims 1 to 9 to generate DNA for sequencing.

17. A use according to claim 16, wherein said sequencing is BAC clone end or BAC clone complete insert sequencing.

18. Use of the method of any one of claims 1 to 9 for genomic DNA sequence extension and cloning.

20. Use of the process of any one of claims 1 to 9 for the Rapid Amplification of 5′- and 3′-Ends of cDNA (RACE).

21. A kit for amplifying target nucleic acids comprising at least one DRT primer, a tagging primer and reagents to effect amplification of nucleic acids.

22. A use according to claim 17, wherein said kit is for human finger printing, genetic testing, bacterial or viral diagnosis, or identification of crop cultivar or hybrid.
